(19) United States
(12) Patent Application Publication
(10) Pub. No.: US 2013/0035752 A1
(43) Pub. Date: Feb. 7, 2013

(54) Dialysis Arterial-Venous Graft

(71) Applicant: Bill Keng Chang, (US)

(72) Inventor: Bill Keng Chang, (US)

(21) Appl. No.: 13/198,671

(22) Filed: Aug. 4, 2011

(51) Int. Cl. A61F 2/06

(52) U.S. Cl. 623/1.13

(57) ABSTRACT

This disclosure relates to an improved arterial-venous graft. In one embodiment, an arterial-venous stent-graft can comprise a tube comprising a first section and a second section, said first section comprising at least fifty percent of said tube. Additionally, the arterial-venous stent-graft can comprise a stent surrounding all of said first section. Additionally, this disclosure relates to a method for placing an arterial-venous stent-graft in a body. Specifically, the method comprises placing an arterial-venous stent-graft in a subcutaneous tunnel, placing a portion of said arterial-venous stent-graft in a vein, wherein said first portion comprises a stent that covers said portion and covers at least fifty percent of said stent-graft, and connecting a second portion of said arterial-venous stent-graft to an artery, further wherein second portion of said arterial-venous stent-graft comprises no stent.

### BACKGROUND

This disclosure relates to an improved arterial-venous graft.

Kidneys in the human body filter waste and excess water from the blood. Presently, people who suffer renal failure require dialysis. Dialysis is used to provide an artificial replacement for dysfunctional kidneys. To perform dialysis, blood is drawn from an artery, filtered, and reintroduced to the body intravenously. People requiring dialysis must have dialysis done on a regular interval through vascular access.

Systems and methods for performing multiple dialysis treatments have evolved, with the eventual introduction of arterial-venous grafts. An arterial-venous graft can be placed under the skin, and connects an artery to a vein, partially diverting blood flow through the stent-graft. During dialysis treatment, a medical professional can draw blood from and reintroduce blood to the body from the arterial-venous graft. The arterial-venous graft can remain under the skin for uses in multiple dialysis treatments. However, common problems exist with presently available arterial-venous grafts. One problem is that present arterial-venous grafts rely on blood pressure and can collapse if there is a poor blood flow, causing complications. Other problems include plaque formation at the venous connection site or needle access sites and kinking or twisting of the arterial-venous grafts.

As such, it would be advantageous to have an improved arterial-venous graft.

### SUMMARY

This disclosure relates to an improved arterial-venous graft, hereinafter referred to as an arterial-venous stent-graft. In one embodiment, an arterial-venous stent-graft can comprise a tube comprising a first section and a second section, said first section comprising at least fifty percent of said tube. Additionally, the arterial-venous stent-graft can comprise a stent surrounding all of said first section.

Additionally, this disclosure relates to a method for placing an arterial-venous stent-graft in a body. Specifically, the method comprises placing an arterial-venous stent-graft in a subcutaneous tunnel, placing a portion of said arterial-venous stent-graft in a vein, wherein said first portion comprises a stent that covers said portion and covers at least fifty percent of said arterial-venous stent-graft, and connecting a second portion of said arterial-venous stent-graft to an artery, further wherein second portion of said arterial-venous stent-graft comprises no stent.

### BRIEF DESCRIPTION OF THE DRAWINGS

FIG. 1 illustrates an arterial-venous stent-graft.

FIG. 2 illustrates arterial-venous stent-graft fully deployed within an artery and a vein of a person.

FIG. 3 illustrates a close up view of stented section of the arterial-venous stent-graft in a vein.

FIG. 4 illustrates a close up view of the non-stented section of arterial-venous stent- graft being connected to an artery.

### DETAILED DESCRIPTION

Described herein is an improved arterial-venous graft. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

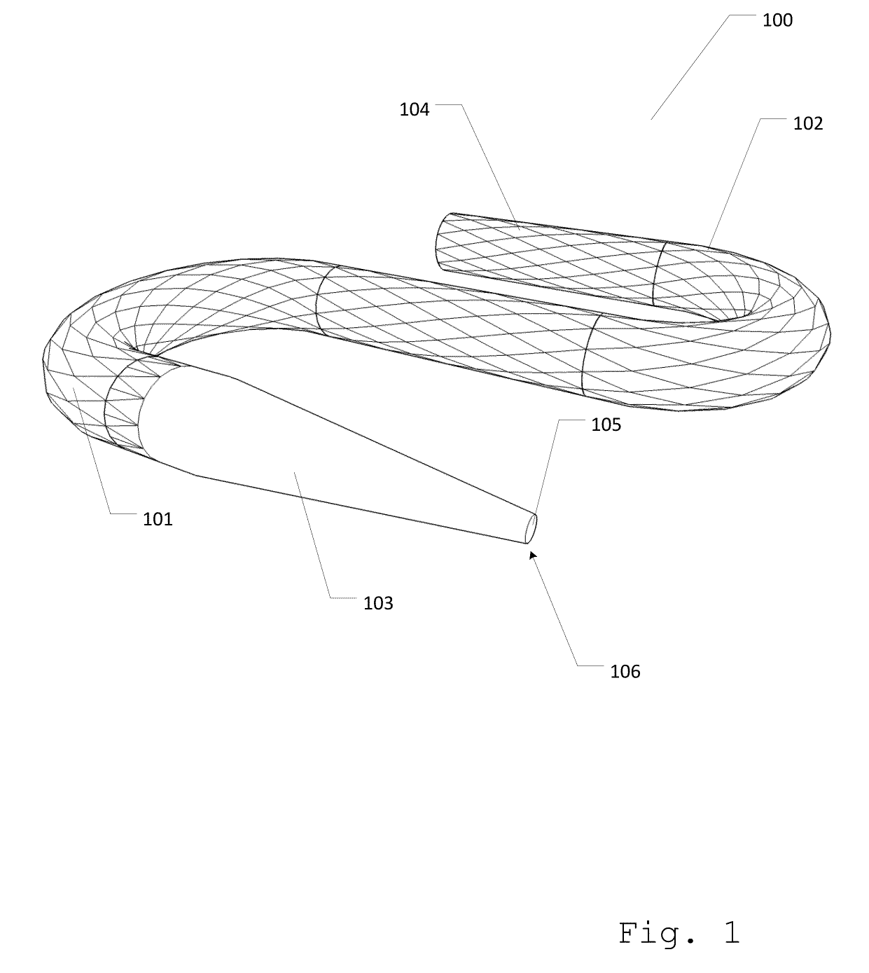

FIG. 1 illustrates an arterial-venous stent-graft (hereinafter referred to as stent-graft 100). Stent-graft 100 can comprise a tube 101 and a stent 102. Tube 101 can comprise two sections, a bare section 103, and a stented section 104. Stented section 104 comprises stent 102 wrapped around tube 101. In one embodiment, stented section 104 comprises at least fifty percent of the length of tube 101. In another embodiment, stented section 104 comprises at least sixty percent of tube 101. In another embodiment, stented section 104 comprises at least seventy percent of tube 101. In another embodiment, stented section 104 comprises at least eighty percent of tube 101. In another embodiment, stented section 104 comprises at least ninety percent of tube 101. Tube 101 can additionally comprise a passageway 105, passing through both sections. Bare section 103 is not surrounded by stent 102. Bare section 103 can be an extrusion made from stented section 104 and can be, in one embodiment, tapered to form a narrow tip at the ending portion 106 of radially tapered surface 104, as shown in FIG. 1. Tube 101 can comprise current available material for vascular graft such as PTFE (polytetrafluoroethylene) or some other vascular graft material. In one embodiment, such stent 102 can comprise nickel-titanium. Stent 102 can serve many different purposes. One purpose is to secure stent-graft 100 within the body. Another purpose is to prevent the collapse of tube 101 after it is inserted into the body.

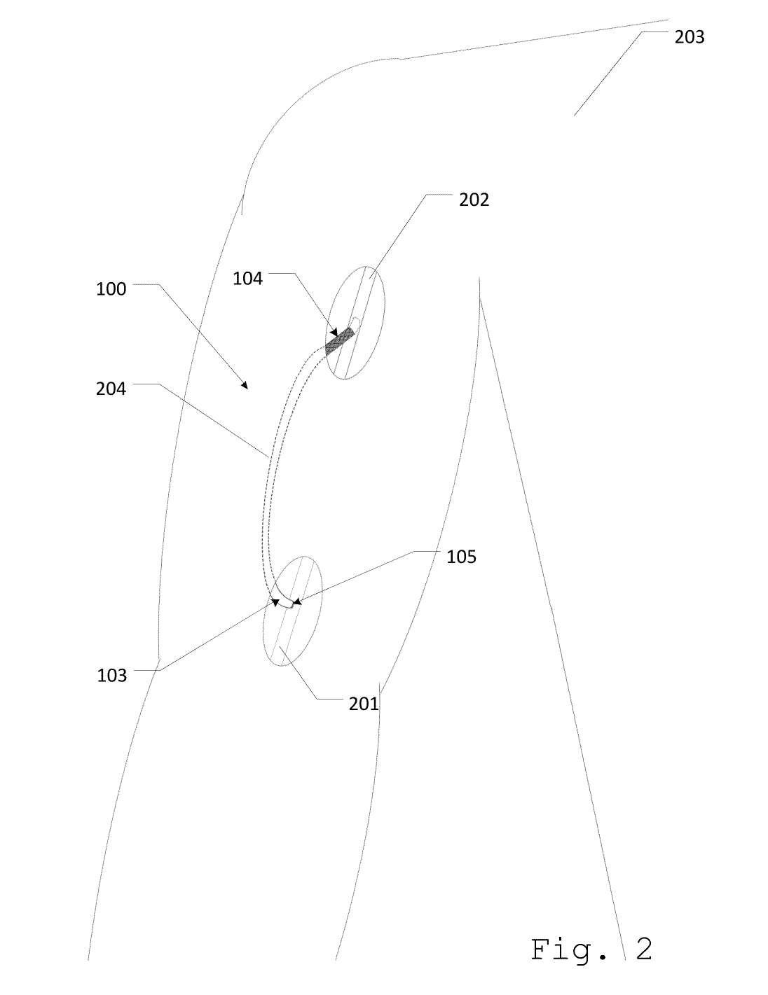

FIG. 2 illustrates stent-graft 100 fully deployed within an arm with connection to an artery 201 and a vein 202 of a person 203. A surgeon can create a one or more incisions in the body. Next, a surgeon can start the process to place stent-graft 100 under the skin of person 203. First, a subcutaneous tunnel 204 can be made inside the arm of person 203. Second, a wire can be introduced within subcutaneous tunnel 204 and enter vein 202. Next, the surgeon can deploy stent-graft 100 over a wire or using any other delivery method known in the art, providing a quick placement of tube device in the subcutaneous tissue of an arm and insertion of tube device into vein 202. Stented section 104 can, in one embodiment, be secured to vein 202 using sutures. Bare non-stented section 103 can be connected to artery 201 using sutures in a typical end-to-side-anastomosis fashion.

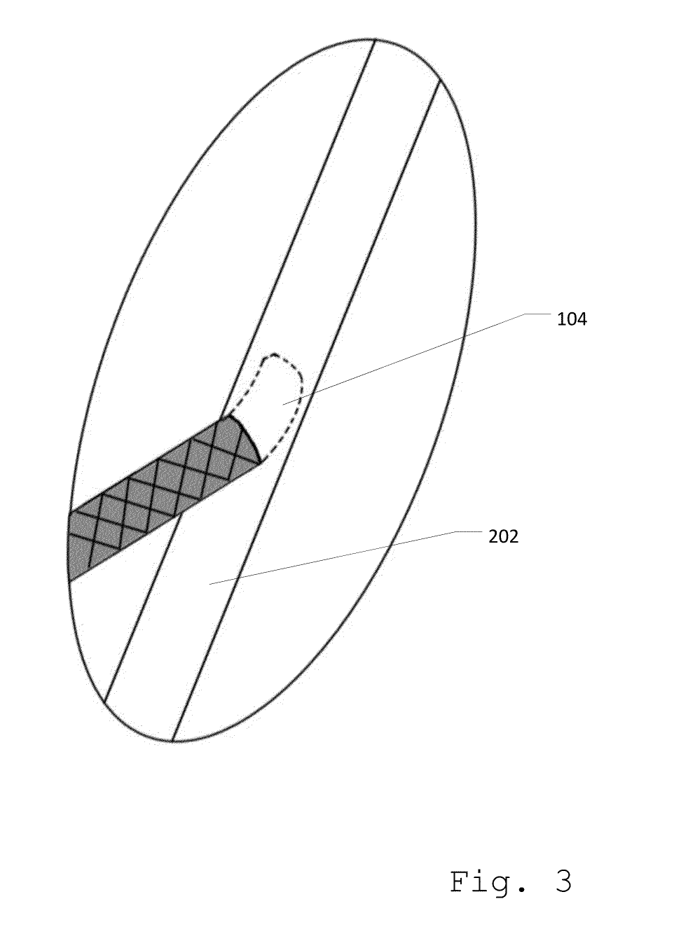

FIG. 3 illustrates a close-up view of stented section 104 in vein 202. Upon deployment, stent expands from a previously collapsed state to a functional deployed or “expanded” state. In such state, a small portion of stented section 104 remains within vein 202, while a predominate portion of stented section 104 remains within subcutaneous tunnel 204. Once deployment system is removed, the surgeon can ensure stented section 104 is firmly within vein 202.

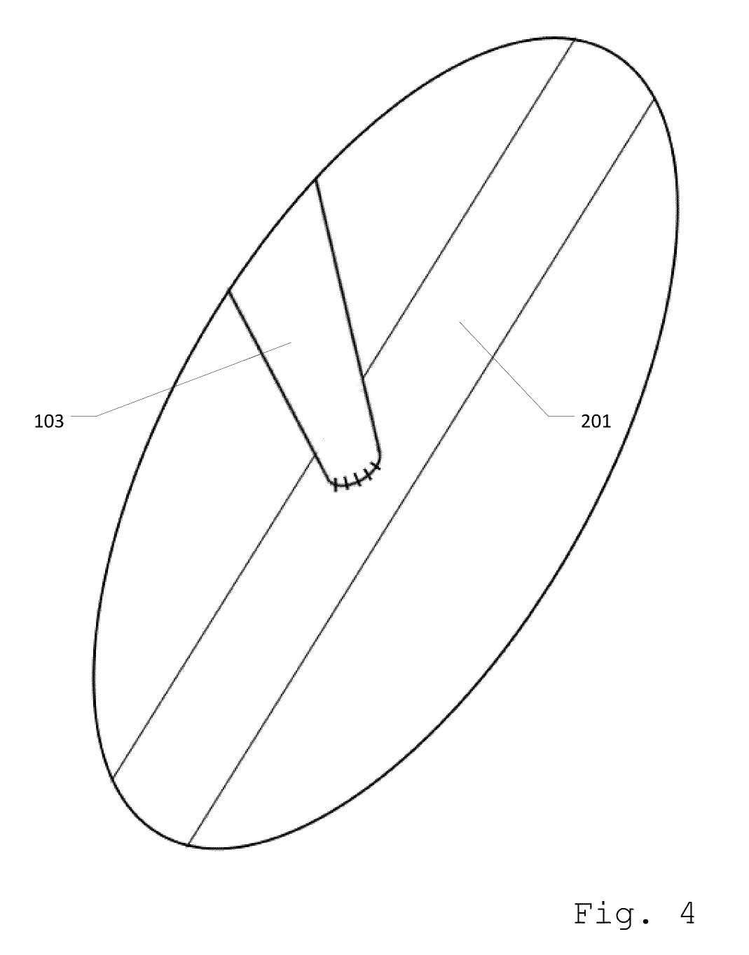

FIG. 4 illustrates a close up view of stent-graft 100 being connected to artery 201. Once the surgeon has attached stent-graft 100 to vein 202, bare section 103 can be sutured to artery 201. Tapering can prevent diverting too much blood supply to stent-graft 100 from artery 201, reducing the chance of complications such as ischemia. Once both ends of stent-graft 100 are secured, incisions can now be closed.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. An arterial-venous stent-graft
a tube comprising a first section and a second section, said first section comprising at least fifty percent of said tube; and
a stent surrounding all of said first section.

2. The arterial-venous stent-graft of claim 1 wherein said first section comprises at least sixty-percent of said tube.

3. The arterial-venous stent-graft of claim 1 wherein said first section comprises at least seventy-percent of said tube.

4. The arterial-venous stent-graft of claim 1 wherein said first section comprises at least eighty-percent of said tube.

5. The arterial-venous stent-graft of claim 1 wherein said first section comprises at least ninety-percent of said tube.

6. The arterial-venous stent-graft of claim 1 wherein said second section comprises a tapered surface.

7. The arterial-venous stent-graft of claim 1 wherein said stent comprises a wire mesh.

8. The arterial-venous stent-graft of claim 1 wherein said tube and said stent can exist in a collapsed state and an expanded state.

9. A method for placing an arterial-venous stent-graft in a body comprising
Placing an arterial-venous stent-graft in a subcutaneous tunnel;
Placing a portion of said arterial-venous stent-graft in a vein, wherein said first portion comprises a stent that covers said portion and covers at least fifty percent of said arterial-venous stent-graft;
Connecting a second portion of said arterial-venous stent-graft to an artery, further wherein second portion of said graft comprises no stent.
Attaching said tapered surface to a one artery, and said arterial-venous stent-graft section to a vein.

10. The method of claim 9 wherein said arterial-venous stent-graft is inserted in vein in a collapsed state.

11. The method of claim 10 wherein said arterial-venous stent-graft is deployed to an expanded state after being inserted in vein.

12. The method of claim 11 wherein said arterial-venous stent-graft is deployed over a wire.

13. The method of claim 9 wherein said arterial-venous stent-graft is connected to said artery by sutures.
